(19) United States
(12) Patent Application Publication
(10) Pub. No.: US 2022/0188816 A1
(43) Pub. Date: Jun. 16, 2022

(54) SYSTEM AND METHOD FOR FACILITATING PAYMENT REQUESTS WITHIN A HEALTH CARE NETWORK

(71) Applicant: Patientory, Inc., Atlanta, GA (US)

(72) Inventor: Chrissa Tanelia McFarlane, Atlanta, GA (US)

(21) Appl. No.: 17/607,226

(22) PCT Filed: Jun. 10, 2019
(86) PCT No.: PCT/US19/36421

§ 371 Date: Oct. 28, 2021

Related U.S. Application Data
(60) Provisional application No. 62/683,513, filed on Jun. 11, 2018.
(60) Provisional application No. 62/683,524, filed on Jun. 11, 2018.
(60) Provisional application No. 62/683,537, filed on Jun. 11, 2018.
(60) Provisional application No. 62/683,556, filed on Jun. 11, 2018.
(60) Provisional application No. 62/683,568, filed on Jun. 11, 2018.

(51) Int. Cl. G06Q 20/38; G16H 10/60

(52) U.S. Cl. CPC G06Q 20/3829; G06Q 20/389; G16H 10/60

(57) ABSTRACT

A system and a method for facilitating payment requests within a health care network are disclosed. The method includes providing a patient interface connected to the health care network. Further, a non-patient interface connected to the health care network is provided. The method further includes receiving a payment request from a payment sender for making a payment to a payment receiver. The payment sender is one and the payment receiver is another of patients and non-patients. Thereafter, the payment request is processed based on at least one of an authentication of the payment receiver and payment details stored in a blockchain database.

### CROSS REFERENCE TO RELATED APPLICATIONS

The present disclosure is related and claims priority under 35 U.S.C. § 1.119(e) to U.S. provisional application No. 62/683,513, entitled SYSTEM AND METHOD FOR MANAGING PAYMENTS FOR ACCESSING PATIENTS INFORMATION; 62/683,524, entitled SYSTEM AND METHOD OF CONTROLLING ACCESS OF A USERS HEALTH INFORMATION STORED OVER A HEALTH CARE NETWORK; 62/683,537, entitled SYSTEM AND METHOD FOR REGULATING A VALUE OF A CRYPTOCURRENCY USED IN A HEALTH CARE NETWORK, 62/683,556, entitled SYSTEM AND METHOD FOR FACILITATING PAYMENT REQUESTS WITHIN A HEALTH CARE NETWORK, and 62/683,568, entitled SYSTEM AND METHOD OF MANAGING ACCESS OF A USERS HEALTH INFORMATION STORED OVER A HEALTH CARE NETWORK, all filed on Jun. 11, 2018, to Chrissa Tanelia McFarlane, the contents of all of which are hereby incorporated by reference in their entirety, for all purposes.

### FIELD OF THE DISCLOSURE

The present disclosure is generally related to a payment system, and more particularly related to a method for facilitating payment requests within a health care network implemented over a blockchain network.

### BACKGROUND

The subject matter discussed in the background section should not be assumed to be prior art merely as a result of its mention in the background section. Similarly, a problem mentioned in the background section or associated with the subject matter of the background section should not be assumed to have been previously recognized in the prior art. The subject matter in the background section merely represents different approaches, which in and of themselves may also correspond to implementations of the claimed technology.

To protect important information, utilizing storage on cloud networks is one approach to provide data redundancy. For sensitive information, the information may be stored in an encrypted form. Blockchain leverages both cloud networks and encryption to define storage of all information in a block wise manner The blocks are added to the blockchain in a linear and chronological order. Various types of information such as patient information, or ledgers or hash values of the information, are stored and hashed in a blockchain database. Currently, the hashes of this information are easily accessible to various entities such as hospitals, insurers, or contract research organizations. Such access to the information may lead to its misuse by the different entities. Therefore, there is a need for a method for controlling access to the patient information and a method of managing payments made for accessing the patient information.

### SUMMARY

In a first embodiment, a computer-implemented method for facilitating payment requests within a health care network includes configuring a patient interface coupled with the health care network for communicating with multiple patients. The computer-implemented method also includes configuring a non-patient interface coupled with the health care network for communicating with multiple non-patients, and receiving a payment request from a payment sender for making a payment to a payment receiver, wherein the payment sender is one and the payment receiver is another of patients and non-patients. The computer-implemented method also includes processing the payment request based on at least one of an authentication of the payment receiver and payment details stored in a blockchain database.

In a second embodiment, a system for facilitating payment requests within a health care network includes a memory circuit storing instructions and one or more processors configured to execute the instructions. The instructions cause the system to configure a patient interface coupled with the health care network for communicating with multiple patients and to configure a non-patient interface coupled with the health care network for communicating with multiple non-patients. The instructions also cause the system to receive a payment request from a payment sender for making a payment to a payment receiver, wherein the payment sender is one and the payment receiver is another of patients and non-patients, and to process the payment request based on at least one of an authentication of the payment receiver and payment details stored in a blockchain database.

In yet other embodiments, a computer-implemented method for processing payment information in a healthcare network, includes receiving a payment information from a remote procedure call component. The computer-implemented method also includes providing the payment information to a payment history blockchain database, creating a hash function for the payment information, and comparing the hash function with existing hash functions in the payment history blockchain database. When the hash function matches with an existing hash function in the payment history blockchain database, the computer-implemented method includes synchronizing, in a syncing node, the payment information with a payment history blockchain database. The computer-implemented method also includes transferring funds from a payment sender to a payment receiver based on the payment information.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g., boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a network connection diagram of a Health Information Exchange (HIE) system, in accordance with various embodiments.

FIG. 2A illustrates a method for symmetric encryption of data in accordance with various embodiments.

FIG. 2B illustrates a method for asymmetric encryption of data, according to various embodiments.

FIG. 3 illustrates a method for hybrid encryption of data, according to various embodiments.

FIG. 4 illustrates a system for storing and accessing data in a health care network, according to various embodiments.

FIG. 5 illustrates a system for storing and accessing data in a health care network implemented over a blockchain network, according to various embodiments.

FIG. 6A illustrates an example of a table showing various example types of information stored in a payment history blockchain database, according to various embodiments.

FIG. 6B illustrates an example of a table showing various example types of information stored in a blockchain database, according to various embodiments.

FIG. 7 illustrates an example of a flowchart showing a method that can be performed by a payment verification module, according to various embodiments.

FIG. 8 illustrates an example of a flowchart showing a method that can be performed by a secure payment module, according to various embodiments.

FIG. 9 illustrates an example of a flowchart showing a method that can be performed by a payment facilitator module, according to various embodiments.

FIG. 10 illustrates an example of a flowchart showing a method that can be performed by a payment blockchain syncing node, according to various embodiments.

FIG. 11 is a block diagram that illustrates a computer system used to perform at least some of the steps and methods in accordance with various embodiments.

### DETAILED DESCRIPTION

Some embodiments of this disclosure, illustrating all its features, will now be discussed in detail. The words “comprising,” “having,” “containing,” and “including,” and other forms thereof, are intended to be open ended in that an item or items following any one of these words is not meant to be an exhaustive listing of such item or items, or meant to be limited to only the listed item or items.

It should also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the particular embodiment of the systems and methods will be described.

Current systems and methods for storing and managing transfer of health information between multiple parties in the healthcare system are often centralized structures subject to hacking, and yet mired in strict security regulations and onerous overhead costs. This state of affairs leads to a lack of efficient and transparent information exchange, to the ultimate detriment of patients and physicians Embodiments as disclosed herein resolve the above technical problem arising in the realm of healthcare data management by implementing a blockchain infrastructure to minimize security breaches and facilitate coordination between multiple entities and organizations, thus improving the health outcomes for patients.

In some embodiments, a blockchain infrastructure as disclosed herein allows the care providers to avoid medication errors, thus reducing the need for duplicate testing. Further, blockchain technology as disclosed herein effectively tracks and timestamps activities related to health information data. Thus, some embodiments provide a robust audit trail that ensures access to all interested and authorized parties to an updated version of a medical record.

Furthermore, in some embodiments, a blockchain network as disclosed herein includes smart contracts configured with universal parameters. Accordingly, patients become the primary intermediaries for sending and receiving health information. Records stored in a blockchain network as disclosed herein are robust to tampering or error, and stored across multiple participating users (e.g., the entire blockchain network). Accordingly, recovery contingencies are unnecessary. Moreover, the transparency of a blockchain network as disclosed herein substantially reduces the number of data exchange integration points and the need for tedious reporting activities.

In some embodiments, a mobile application installed in client devices allow users to interact with the blockchain network and access features such as messaging, and access updated and accurate health information. Further, some embodiments provide tracking applications and other activity trackers to enable doctors, care providers, and other parties in the blockchain network to communicate on a single, easy to use platform. Furthermore, in some embodiments, artificial intelligence, machine learning, neural networks, and other nonlinear algorithms are incorporated to store and manage data in the blockchain network.

Some embodiments provide the ability for patients and other users of the blockchain network to access tokens from an external blockchain to convert into a supported cryptocurrency for access and use of storage features.

Embodiments of the present disclosure will be described more fully hereinafter with reference to the accompanying drawings in which like numerals may represent like elements throughout the several figures, and in which various example embodiments are shown. Embodiments may, however, be embodied in many different forms and should not be construed as limited to the embodiments set forth herein. The examples set forth herein are non-limiting examples and are merely examples among other possible examples.

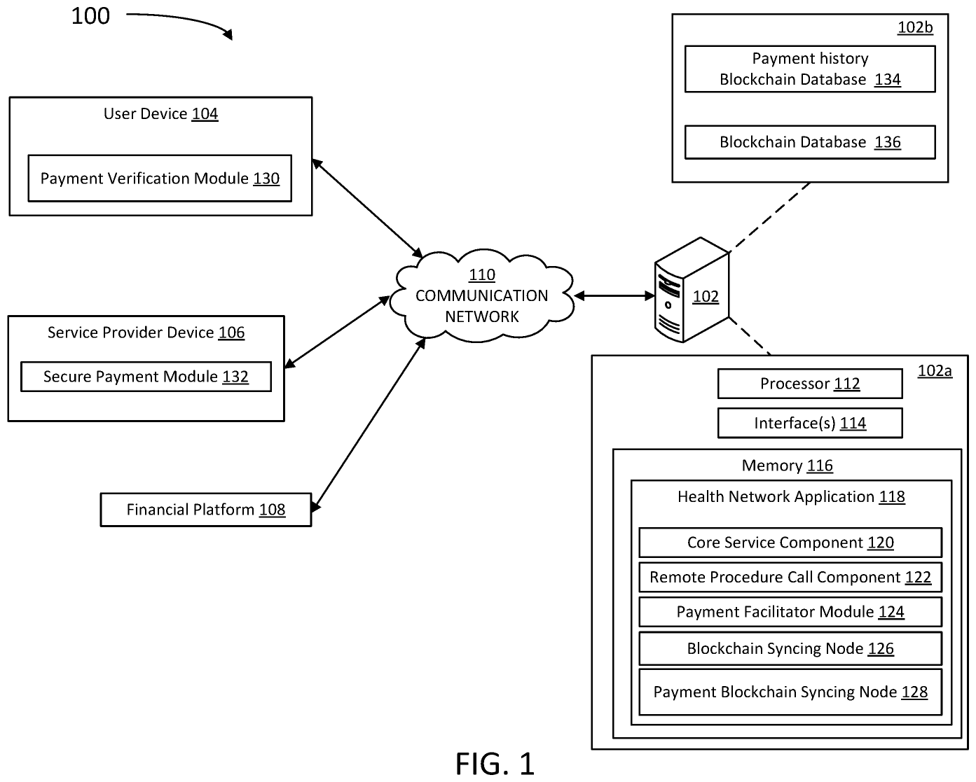

FIG. 1 illustrates a network connection diagram 100 of a Health Information Exchange (HIE) system 102 for facilitating payment requests within a health care network. The HIE system 102 may include one or more user interfaces. In various embodiments, the one or more user interfaces may be accessed by one or more users via one or more user devices; however a single user device 104 is illustrated for simplicity, multiple such user devices could be used. The HIE system 102 may be connected with the user device 104, a service provider device 106, and a financial platform 108, through a communication network 110.

The communication network 110 may be a wired and/or a wireless network. The communication network 110, if wireless, may be implemented using communication techniques such as, for example, Visible Light Communication (VLC), Worldwide Interoperability for Microwave Access (WiMAX), Long Term Evolution (LTE), Wireless Local Area Network (WLAN), Infrared (IR) communication, Public Switched Telephone Network (PSTN), Radio waves, and other communication techniques known in the art.

The HIE system 102 may include a group of components 102a for facilitating the payment requests within the health care network. The group of components 102a may include a processor 112, interface(s) 114, and a memory 116. The memory 116 may include a health network application 118. The health network application 118 may include a core service component 120, a Remote Procedure Call (RPC) component 122, a payment facilitator module 124, a blockchain syncing node 126 (e.g., for a private blockchain network—Quorum- or a public blockchain network), and a payment blockchain syncing node 128.

The processor 112 may execute an algorithm stored in the memory 116 for facilitating the payment requests within the health care network. The processor 112 may also be configured to decode and execute any instructions received from one or more other electronic devices or server(s). The processor 112 may include one or more general purpose processors (e.g., microprocessors) and/or one or more special purpose processors (e.g., digital signal processors or System On Chips (SOCs), Field Programmable Gate Arrays (FPGAs) processor, or Application-Specific Integrated Circuits (ASICs)). The processor 112 may be configured to execute one or more computer-readable program instructions, such as program instructions to carry out any of the functions described in this description.

The interface(s) 114 may help an operator to interact with the HIE system 102. The interface(s) 114 may either accept inputs from users or provide outputs to the users, or may perform both the actions. In various embodiments, a user can interact with the interface(s) 114 using one or more user-interactive objects and devices. The user-interactive objects and devices may include user input buttons, switches, knobs, levers, keys, trackballs, touchpads, cameras, microphones, motion sensors, heat sensors, inertial sensors, touch sensors, or a combination of the above. Further, the interface(s) 114 may either be implemented as a Command Line Interface (CLI), a Graphical User Interface (GUI), a voice interface, or a web-based user-interface.

The memory 116 may include, but is not limited to, fixed (hard) drives, magnetic tape, floppy diskettes, optical disks, Compact Disc Read-Only Memories (CD-ROMs), and magneto-optical disks, semiconductor memories, such as ROMs, Random Access Memories (RAMs), Programmable Read-Only Memories (PROMs), Erasable PROMs (EPROMs), Electrically Erasable PROMs (EEPROMs), flash memory, magnetic or optical cards, or other type of media/machine-readable medium suitable for storing electronic instructions.

In accordance with various embodiments, several users may interact with the HIE system 102, using the user device 104. The user device 104 may include a payment verification module 130. Although a single user device has been illustrated, several user devices could similarly be connected to the communication network 110. Further, each of the user devices may have a device ID. In various embodiments, the device ID may be a unique identification code such as an (International Mobile Equipment Identity) IMEI code or a product serial number. It should be noted that a user may use a single user device or multiple user devices. Further, multiple users may use a single user device or multiple user devices. Further, the one or more users may receive and/or provide healthcare related products and services. The one or more users may include, for example, patients, family and friends of the patients, hospitals, physicians, nurses, specialists, pharmacies, medical laboratories, testing centers, insurance companies, or Emergency Medical Technician (EMT) services.

The user device 104 may be a stationary device, a portable device, or a device accessed remotely. The user device 104 may be, but is not limited to, a computer, a laptop, a tablet, a mobile phone, a smartphone, or a smart watch. In various embodiments, the user device 104 may include an imaging device that may be configured to capture a visual graphical element, the visual graphical element such as, but not limited to, a barcode, text, a picture, or any other form of graphical authentication indicia. In various embodiments, the barcode may be one-dimensional or two-dimensional. Further, the imaging device may include a hardware and/or software element. In various embodiments, the imaging device may be a hardware camera sensor that may be operably coupled to the user device 104. In various embodiments, the hardware camera sensor may be embedded in the user device 104. In various embodiments, the imaging device may be located external to the user device 104. In various embodiments, the imaging device may be connected to the user device 104 wirelessly or via a cable. It should be noted that image data of the visual graphical element may be transmitted to the user device 104 via the communication network 110.

In accordance with various embodiments, the imaging device may be controlled by applications and/or software(s) configured to scan a visual graphical code. In various embodiments, a camera may be configured to scan a QR code. Further, the applications and/or software(s) may be configured to activate the camera present in the user device 104 to scan the QR code. In various embodiments, a processor natively embedded in the user device 104 may control the camera. In another case, the imaging device may include a screen capturing software (for example, screenshot) that may be configured to capture and/or scan the QR code on a screen of the user device 104.

In accordance with various embodiments, a service provider may use the service provider device 106. In various embodiments, a hospital, an insurer, or a pharmaceutical company may operate the service provider device 106. The service provider device 106 may include a secure payment module 132. Further, the service provider device 106 may include an interface. The service provider device 106 may be, for example, a desktop, a smart phone, tablet, and a phablet. In various embodiments, the financial platform 108 may verify and facilitate changes in balance of subscriber's and patient's account as well as payments to a patient network host.

In accordance with various embodiments, a group of databases 102b may be connected to the HIE system 102. In various embodiments, the group of databases 102b may be implemented over a blockchain network (such as a PTOYNet blockchain network or a PTOYNet EthereumTM blockchain network), and may be present as different databases installed at different locations. The group of databases 102b may include a payment history blockchain database 134 and a blockchain database 136, for a private blockchain network (e.g., Quorum) or a public blockchain network. The group of databases 102b may be configured to store data belonging to different users and data desired for functioning of the HIE system 102. Different databases are used in present case; however, a single database may also be used for storing the data. Usage of the different databases may also allow segregated storage of different data and may thus reduce time to access desired data. In various embodiments, the data may be encrypted, time-dependent, piece-wise, and may be present as subsets of data belonging to each user. For example, the data may represent the results of a medical test amongst multiple medical tests.

In accordance with various embodiments, the group of databases 102b may operate collectively or individually. Further, the group of databases 102b may store data as tables, objects, or other data structures. Further, the group of databases 102b may be configured to store data retrieved or processed by the HIE system 102. The data may include, but is not limited to, patient medical history, medical charts, medications, prescriptions, immunizations, test results, allergies, insurance provider(s), or billing information. Further, the data may be time-dependent and piece-wise. Further, the data may represent a subset of data for each patient. Further, the data may be securely stored. In various embodiments, the data may be encrypted.

In accordance with various embodiments, information stored in the group of databases 102b may be accessed based on users' identities and/or the users' authorities. The users' identities may be verified in one or more ways such as, but not limited to, bio-authentication (or biometric authentication), password or PIN information, user device registrations, a second-level authentication, or a third-level authentication. In various embodiments, the users' identities may be verified by the HIE system 102. Information provided by the users in real-time may be used, by the HIE system 102, to confirm the users' identities. In an example, the users' identities may be verified using, for example, a name, a password, one or more security questions, or a combination thereof. In various embodiments, a user may be identified using an encryption key and/or a decryption key.

In accordance with various embodiments, the data stored in the group of databases 102b may be accessed at different levels, for example using a first level subsystem and a second level subsystem. In various embodiments, a user may directly access the first level subsystem. To access data stored in the second level subsystem, the second level subsystem may be accessed through the first level subsystem. It should be noted that the communication between the first level subsystem and the second level subsystem may be encrypted. In an example, the second level subsystem may be implemented over a blockchain network such as, for example, a PTOYNet blockchain network or a PTOYNet EthereumTM based blockchain network. In various embodiments, the PTOYNet Ethereum^{TM} blockchain network may be used to implement smart contracts.

In accordance with various embodiments, a primary care physician may input data into the HIE system 102 using the user device 104. The data may be processed by the first level subsystem and the second level subsystem. This may be done successively. The data may be stored on the first level subsystem and/or the second level subsystem of the HIE system 102. This may be done successively. The data may include, but is not limited to, one or more instructions to a patient to see a physician specialist. Further, the data may be stored in one or more blockchains of the second level subsystem. The patient may be able to access the data relating to the patient's care provided by the primary care physician. This may be done successively. The patient may be able to retrieve the data using the user device 104 of the patient. This may be done successively.

In accordance with various embodiments, the patient may communicate with the physician specialist using the HIE system 102. It should be noted that the physician specialist may be able to access the data of the patient from the first level subsystem and/or the second level subsystem. Further, the physician specialist may be able to communicate with the patient. It should be noted that all (or substantially all) communications between the primary care physician, the physician specialist, and the patient may be stored and may be accessible on a blockchain network (such as a PTOYNet blockchain network or a PTOYNet Ethereum™ blockchain network).

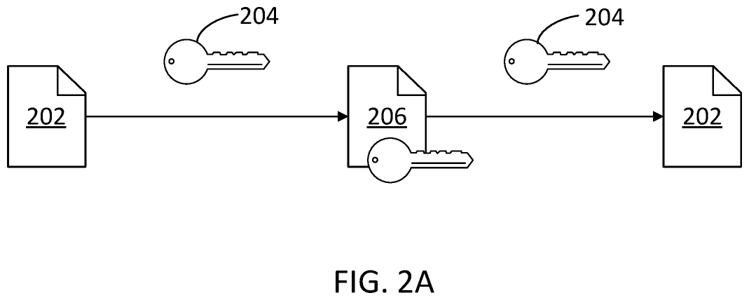

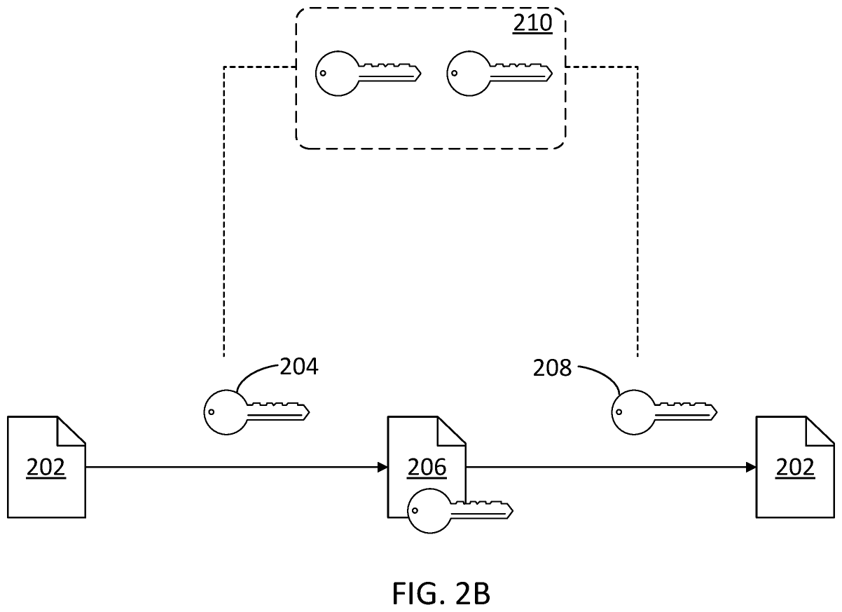

FIG. 2A illustrates a method for symmetric encryption of data, in accordance with various embodiments. Original data 202 may be encrypted using a key 204 to obtain an encrypted data 206. The encrypted data 206 may be decrypted using the key 204 to obtain back the original data 202. It should be noted that encryption and decryption of the data may be performed using a same key. Further, one or more parties involved in a communication may have the same key to encrypt and decrypt the data.

FIG. 2B illustrates a method for asymmetric encryption of data, in accordance with various embodiments. Original data 202 may be encrypted using a key 204 to obtain encrypted data 206. The encrypted data 206 may be decrypted using another key 208 to obtain the original data 202. It should be noted that encryption and decryption of the data may be performed using different keys e.g., a key pair 210.

In some embodiments, the steps illustrated in FIGS. 2A-B may be initiated by users who generate a new profile on the blockchain network. Private keys may be stored in decentralized and distributed hashes through the blockchain network. In some embodiments, the steps illustrated in FIGS. 2A-B may be partially performed in either one of devices 104 and 106, or in HIE system 102 and financial platform 108. For example, in some embodiments, HIE system 102 may install a software development kit (SDK) or a key generator application in user device 104 or in service provider device 106 to perform at least some of the steps illustrated in FIGS. 2A-B. Likewise, keys 204, 208, and key pair 210 may be stored in a memory of either one of devices 104, 106, or in HIE system 102, or financial platform 108, or in an associated database (e.g., any one of databases 102b).

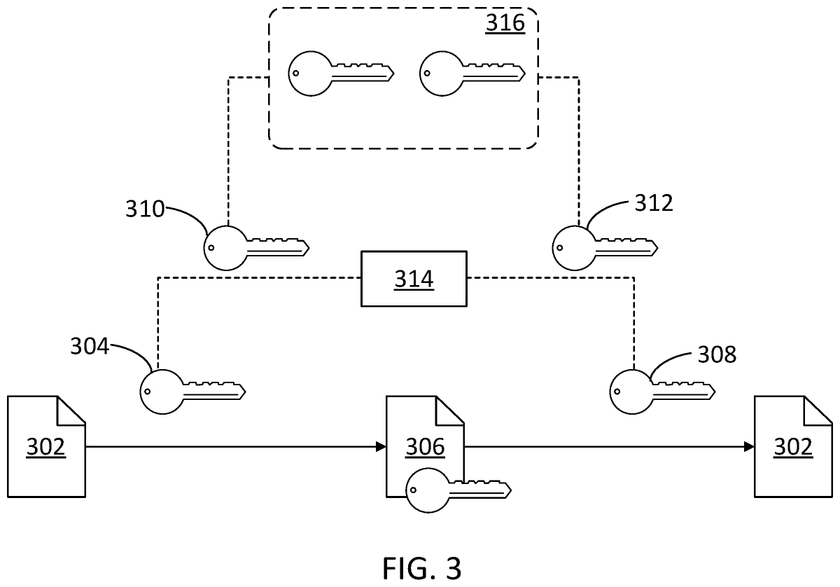

FIG. 3 illustrates a method for hybrid encryption of data, in accordance with various embodiments. Both symmetric encryption and asymmetric encryption techniques may be used in tandem. For example, the symmetric encryption technique may be used to encrypt data 302 using a symmetric key 304 for producing encrypted data 306. The encrypted data 306 may be decrypted using another symmetric key 308 for obtaining data 302. Further, a public key 310 may be used to encrypt the symmetric key 304 and a private key 312 may be used to encrypt the symmetric key 308, stored as an encrypted key 314. The public key 310 and the private key 312 may form a key pair 316.

In some embodiments, the steps illustrated in FIG. 3 may be initiated by users who generate a new profile on the blockchain network. Private keys may be stored in decentralized and distributed hashes through the blockchain network. In some embodiments, the steps illustrated in FIG. 3 may be partially performed in either one of devices 104 and 106, or in HIE system 102 and financial platform 108. For example, in some embodiments, HIE system 102 may install a software development kit (SDK) or a key generator application in user device 104 or in service provider device 106 to perform at least some of the steps illustrated in FIG. 3. Likewise, keys 204, 208, and key pair 210 may be stored in a memory of either one of devices 104, 106, or in HIE system 102, or financial platform 108, or in an associated database (e.g., any one of databases 102b).

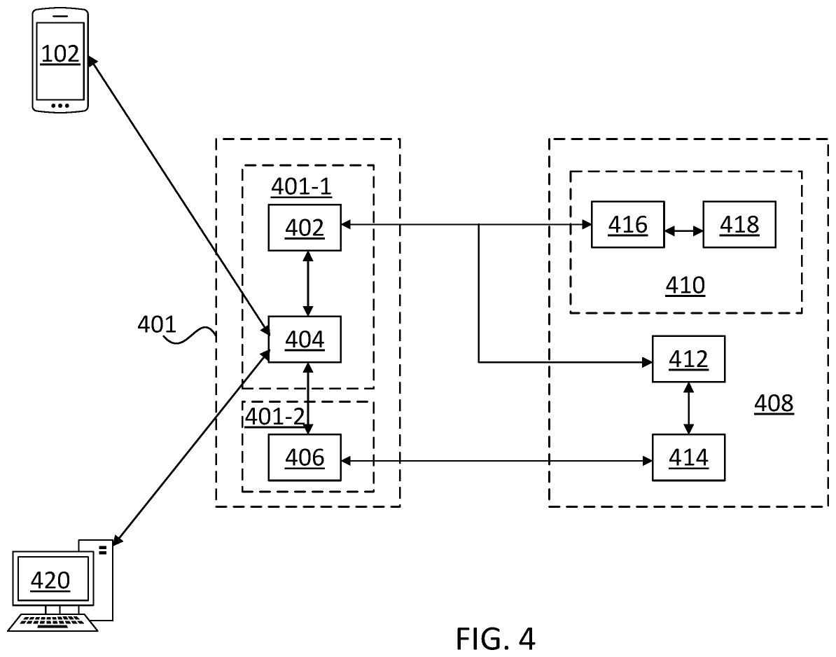

FIG. 4 illustrates a system 401 for storing and accessing data in a health care network, according to various embodiments. A first level subsystem 401-1 may include a core service component 402 and a Remote Procedure Call (RPC) component 404. A second level subsystem 401-2 may include a blockchain node 406. Blockchain node 406 may be a public node or a private node in a blockchain network having a layer over a public blockchain network, enabling the private node to perform private transactions via consensus algorithms (e.g., a Quorum blockchain node). In various embodiments, first level subsystem 401-1 may include the core service component 402, and second level subsystem 401-2 may include the RPC component 404 and the blockchain node 406. Further, the core service component 402 of first level subsystem 401-1 may be present in communication with third-party servers and databases of a hospital computing network 408. The hospital computing network 408 may include a file system module 410, an EHR synchronization service 412, and a blockchain node 414 (e.g., a Quorum blockchain node). Further, the file system module 410 may include a file system manager 416 and a file system node 418. The blockchain node 406 of second level subsystem 401-2 may communicate with the blockchain node 414 of the hospital computing network 408. Patients may access the health care network for storing data through the user device 104, and a representative of a hospital may access the health care network through another user device 420.

In accordance with various embodiments, the representative of the hospital may want to synchronize Electronic Health Record (EHR) data of a patient, e.g., by using corresponding blockchain hashes. Successively, first level subsystem 401-1 and second level subsystem 401-2 may ask the patient for permission to allow a representative of the hospital to store the EHR data of the patient, through the file system module 410. Based at least on the permission granted by the patient, a signed transaction may be created to confirm the permission of the hospital to store the EHR data. Further, the signed transaction may activate a smart contract that may add hospital identification information such as a blockchain address to a list of permitted users. In some embodiments, the signed transaction and the smart contract are stored in file system module 410.

Further, the signed transaction may be transmitted from the user device 104 to the RPC component 404 of the first level subsystem and/or the second level subsystem. The RPC component 404 may communicate the signed transaction to the blockchain node 406 of the second level subsystem. This may be done successively. The blockchain node 406 may activate one or more smart contracts. This may be done successively. Thereafter, the blockchain node 406 may revise a state of one or more blockchains.

Further, based at least on the permission granted by the patient, the EHR synchronization service may obtain a list of patients from the RPC component 404. Further, the EHR synchronization service may confirm whether the patient has granted permission. Based at least on the permission, the first level subsystem and the second level subsystem may obtain the EHR data and may calculate a hash function for the EHR data. The HIE server 106 may match the hash function of the EHR data with a hash function for the patient blockchain on the blockchain node 406 of the second level subsystem. This may be done successively. Thereafter, if the hash function of the EHR data matches with the hash function for the patient blockchain on the blockchain node 406 of the second level subsystem, the EHR data of the patient may remain unchanged.

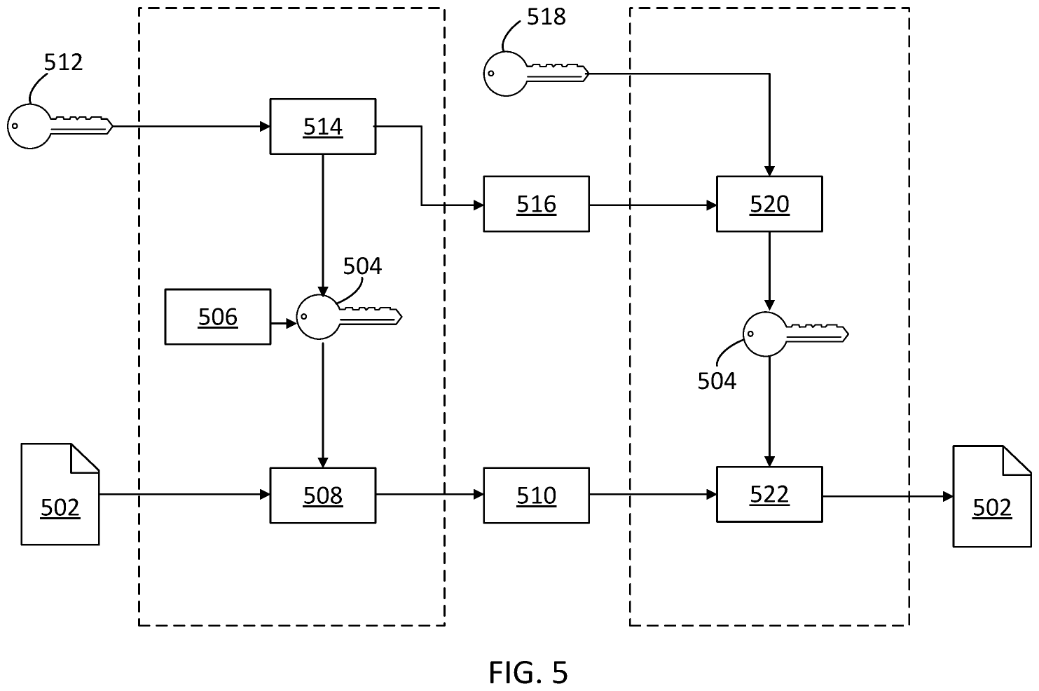

FIG. 5 illustrates a system for storing and accessing data in a health care network implemented over a blockchain network as disclosed herein (cf. FIGS. 1 and 4). The HIE system 102 may execute an application for determining permission from the user for obtaining EHR data 502. In various embodiments, if the user grants the permission, the HIE system 102 may obtain the EHR data 502 for calculating a hash function for the EHR data 502. Further, the HIE system 102 may match the hash function of the EHR data 502 with a hash function for the user blockchain on the blockchain node of the second level sub-system. In various embodiments, if the two hash functions match, there is no change to the user's EHR data 502. In various embodiments, if the two hash functions do not match, the HIE system 102 may generate a random string, e.g., secret key 504, through a random key generator 506. The secret key 504 may be used for Advanced Encryption Standard (AES) encryption of the EHR data 502, in an AES encryptor 508, for generating encrypted EHR data 510.

In various embodiments, the secret key 504 may then be encrypted by, for example, a Rivest-Shamir-Adleman (RSA) public key 512 of the patient, in an RSA encryptor 514, to generate an encrypted secret key 516. The HIE system 102 may further send the encrypted EHR data 510 to the core service component 120 for forwarding the data to the file system manager 416 of the hospital computing network 408 for storage. Further, the file system manager 416 may send a file system hash function to the core service component 120 for further sending the file system hash function to EHR synchronization service 412. The EHR synchronization service 412 may further update the patient smart contract with the new file system hash function, the encrypted random key, a hash function of the unencrypted file, and file name

In accordance with various embodiments, a hospital representative, such as a doctor or a hospital administration, may want to view the EHR data 502. In such a scenario, the user may first send a signed transaction to an RPC component 122 for granting permission to the hospital representative to view the EHR data 502. Once the permission is granted, the signed transaction may be added to the blockchain node 414 and a new smart contract will be created for a blockchain corresponding to the hospital representative. After adding the signed transaction, the hospital representative may be able to view the EHR data 502 of the user on a device.

In accordance with various embodiments, in order to view the EHR data 502 on the device, the HIE system 102 may collect the encrypted EHR data 510 from the user's blockchain and may decrypt the encrypted EHR data 510 using patient's RSA private key 518. The HIE system 102 may decrypt the encrypted secret key 516, in an RSA decryptor 520, using an RSA private key of the hospital representative. The encrypted EHR data 510 may be decrypted using the RSA public key 512 of the hospital representative, in an AES decryptor 522. This may be done successively. Further, the HIE system 102 may load the decrypted EHR data 502 to the smart contract previously created for the hospital representative.

After loading the decrypted EHR data to the smart contract, the RPC component 122 may obtain the signed transaction from the patient's user device and transmit the signed transaction to the blockchain node 406 of the second level subsystem. The blockchain node 406 may confirm ownership of the signed transaction and may execute the smart contract for the hospital representative to view the user's data. This may be done successively.

In accordance with various embodiments, the patient may decline permission for the hospital representative to have access to the EHR data 502. In such a scenario, the user through a user device may send a signed transaction revoking permission to the RPC component 122. The RPC component 122 may forward the signed transaction to the blockchain node 406 of the second level subsystem. This may be done successively. The blockchain node 406 may confirm ownership of the signed transaction and may delete the smart contract previously created to allow the hospital representative to have access to the patient's EHR data 502. This may be done successively.

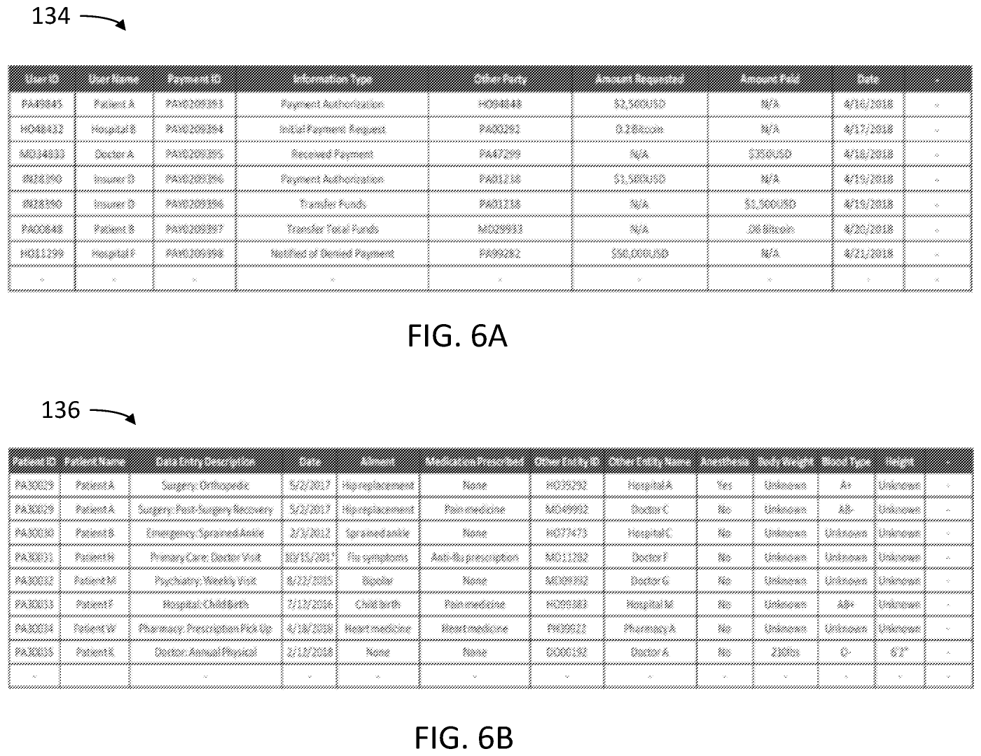

FIG. 6A illustrates at least partially a payment history blockchain database 134, according to some embodiments. In accordance with various embodiments, payment history blockchain database 134 may be configured to store payment histories of one or more parties. The one or more parties may be patients, doctors, hospitals, or insurers. In some embodiments, payment history blockchain database 134 may store a user ID, a user name, payment ID, information type, other party, an amount requested, amount paid, or date. For example, the information type may be, for example, a payment request, transfer of funds, or denial of payment. Further, an amount requested may be, for example, 2000USD or 0.001 bitcoin.

FIG. 6B illustrates at least partially a blockchain database 136, according to some embodiments. Blockchain database 136 may be configured to store healthcare related information of patients. In some embodiments, blockchain database 136 may store a patient ID, a patient name, data entry description, date, ailment, medication prescription, other entity ID or anesthesia. Further, the blockchain database 136 may store one or more parameters of the patient such as body weight, blood type, or height. The blockchain database 136 may be used by patients or the parties to verify that the payments are accurate. In an example, if a patient received a request from a hospital to pay for a liver transplant and the liver transplant never occurred, then the patient may check the blockchain database 136 to confirm that procedure has not happened and the payment is inaccurate.

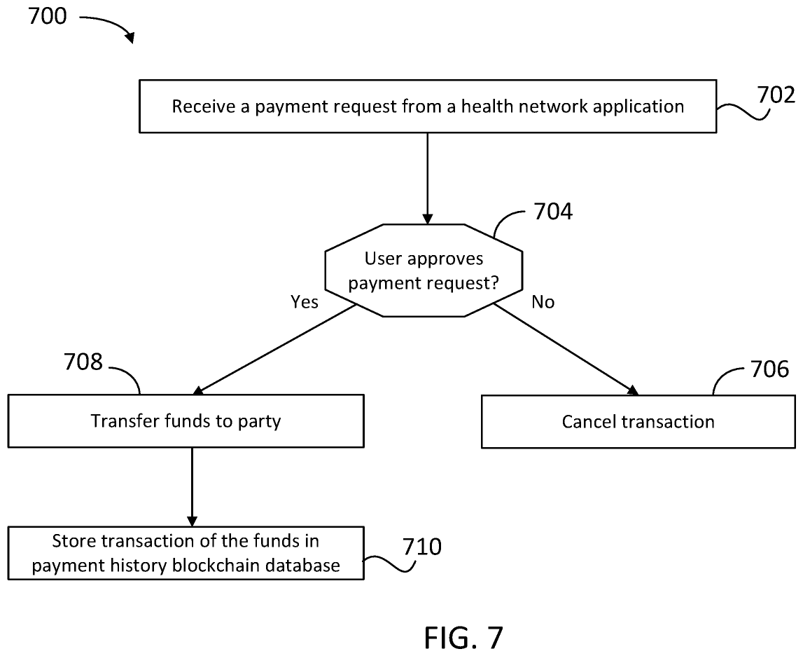

FIG. 7 illustrates an example of a flowchart 700 showing a method that can be performed by a payment verification module 130, in accordance with various embodiments. Functioning of the payment verification module 130 will now be explained with reference to the flowchart 700 shown in FIG. 7. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

The payment verification module 130 may receive a payment request from the health network application 118, at step 702. The payment request may include information such as, for example, but not limited to, details of a person requesting payment and a purpose of the payment request, such as surgery or a doctor's appointment. In various embodiments, the payment request may be made by a hospital or an insurer. It should be noted that the user may receive the payment request via a user interface. In various embodiments, the payment verification module 130 may determine whether the user approves the payment request, at step 704. This may be done successively. In various embodiments, if the user does not approve the payment request, then the payment verification module 130 may cancel transaction, at step 706. It should be noted that based upon the cancellation, a notification may be sent to a party requesting the payment. In various embodiments, if the user approves the request, the payment verification module 130 may transfer funds to the party, at step 708. The funds may be transferred to a mobile wallet of the user. Transaction of the funds may be stored in the payment history blockchain database 134, at step 710. It should be noted that such transaction of the funds may be tracked by the health network application 118. This may be done successively.

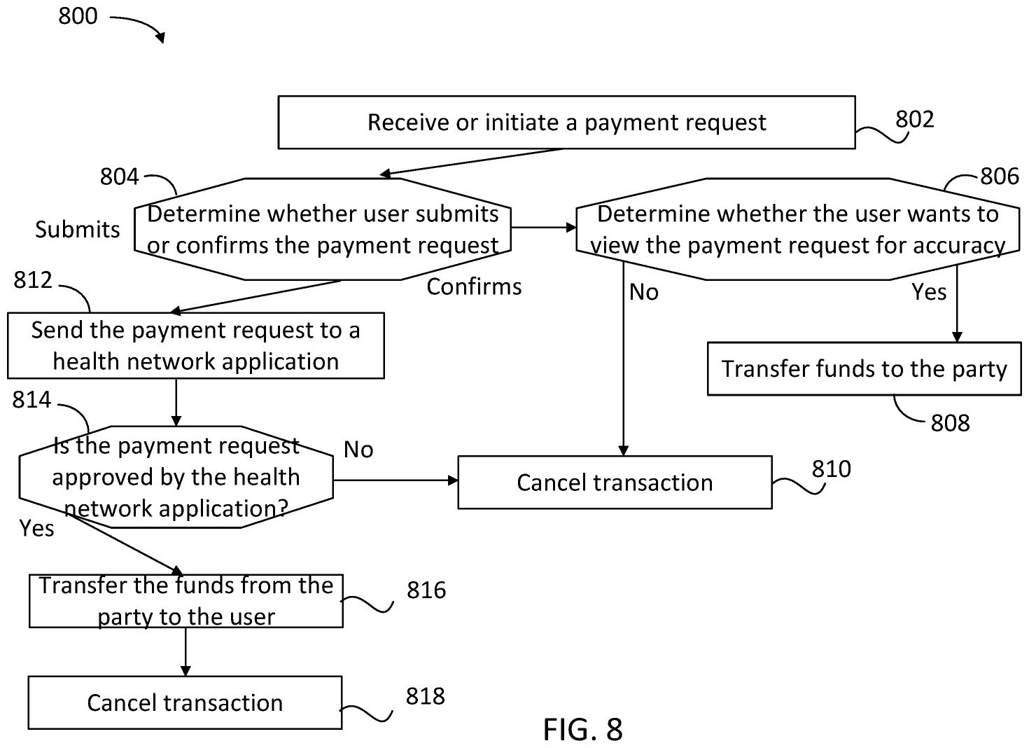

FIG. 8 illustrates an example of a flowchart 800 showing a method that may be performed by the secure payment module 132, according to various embodiments. Functioning of the secure payment module 132 will now be explained with reference to the example flowchart 800 shown in FIG. 8. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the secure payment module 132 may receive or initiate the payment request, at step 802. The payment request may be received or initiated using a service provider interface (e.g., a non-patient interface). It should be noted that information related to the payment request may be filled and submitted by the user to the health network application 118. The secure payment module 132 may determine whether the user submits or confirms the payment request, at step 804. This may be done successively. In various embodiments, if the user confirms the payment request, the secure payment module 132 may determine whether the user wants to view the payment request for accuracy, at step 806. In various embodiments, if the user determined that the payment request is accurate, then the secure payment module 132 may transfer the funds to the party, at step 808. The funds may be transferred via the mobile wallet of the user.

In accordance with various embodiments, the party may be an insurance company. The insurance company may review the information of the payment request, stored in the payment history blockchain database 134. In various embodiments, the insurance company may view a portion of the user's medical records in the blockchain database 136 to determine whether the payment request is accurate. If the payment request is accurate, then the insurance company may transfer the funds to the party. In various embodiments, if the user determined that the payment request is not accurate, then the secure payment module 132 may cancel the transaction, at step 810. It should be noted that based on the cancellation, the secure payment module 132 may send a notification to the user.

If the user submits the payment request, then the secure payment module 132 may send the payment request to the health network application 118, at step 812. This may be done successively. The secure payment module 132 may determine whether the payment request is approved by the health network application 118, at step 814. This may be done successively. In various embodiments, if the payment request is approved by the health network application 118, then the secure payment module 132 may transfer the funds from the party to the user via the health network application 118, at step 816. In various embodiments, if the payment request is not approved by the health network application 118, then the secure payment module 132 may cancel the transaction, at step 818. Further, a notification may be sent to the user that the payment has been denied. It should be noted that the transaction and the payment requests may be tracked and stored in the payment history blockchain database 134.

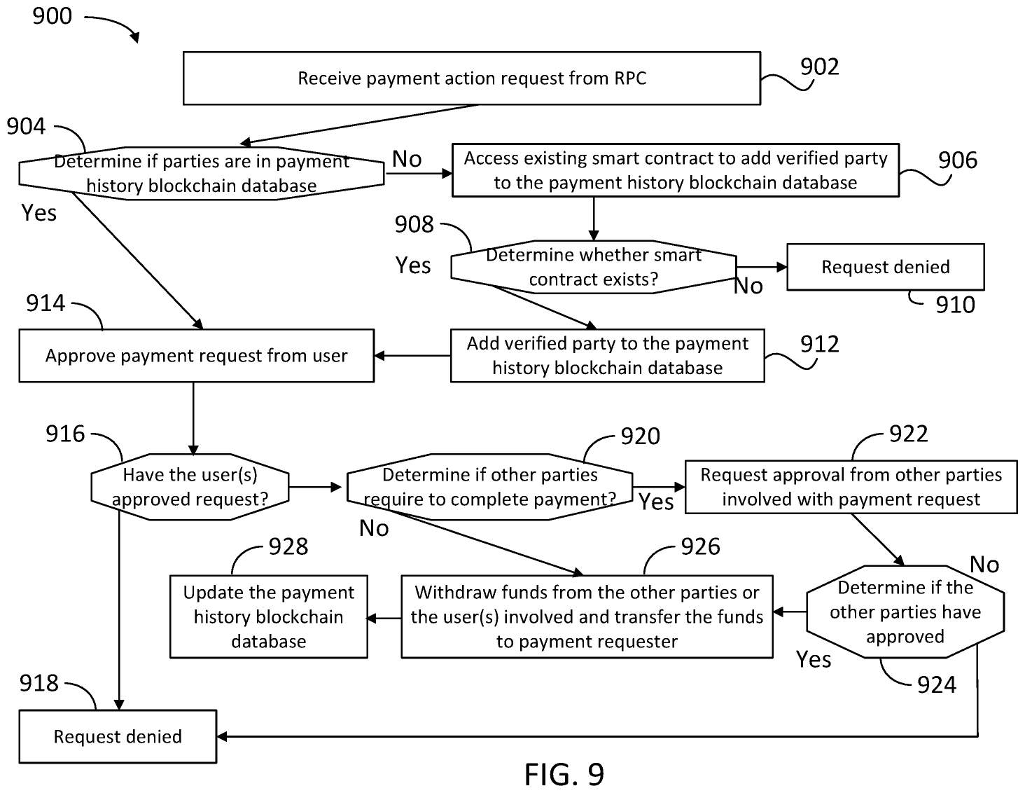

Functioning of the payment facilitator module 124 will now be explained with reference to the example flowchart 900 shown in FIG. 9. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In accordance with various embodiments, the payment facilitator module 124 may receive a request (e.g., a payment request) from the RPC component 122, at step 902. In various embodiments, the payment facilitator module 124 may analyze the payment action request to establish what is being requested and which parties are involved. Further, the payment facilitator module 124 may determine whether the parties are present in the payment history blockchain database 134, at step 904. In various embodiments, if the parties are not present in the payment history blockchain database 134, the payment facilitator module 124 may access existing smart contracts to add verified party to the payment history blockchain database 134, at step 906. In various embodiments, the smart contract may be generated by the RPC component 122 and the core service component 120.

In accordance with various embodiments, the payment facilitator module 124 may determine whether the smart contract exists, at step 908. This may be done successively. In various embodiments, if the smart contract does not exist, then the payment facilitator module 124 may deny the payment request, at step 910. In another case, if the smart contract exists, then the payment facilitator module 124 may add the verified party to the payment history blockchain database 134, at step 912. The verified party may be an insurer, a patient, or a hospital.

In accordance with various embodiments, if the parties are present in the payment history blockchain database 134, then the payment facilitator module 124 may request an approval from the user involved with the payment request, at step 914. This may be done successively. In accordance with various embodiments, the payment facilitator module 124 may determine whether a patient has approved the request, at step 916. This may be done successively. In accordance with various embodiments, if the patient has not approved the request, the payment facilitator module 124 may deny the payment request, at step 918. It should be noted that the party may be notified by the payment facilitator module 124.

In accordance with various embodiments, if the patient approves the request, then the payment facilitator module 124 may determine whether other parties require for completing the payment, at step 920. This may be done successively. In various embodiments, if the other parties are required to complete the payment, then the payment facilitator module 124 may request other parties' approval who are involved with the payment request, at step 922. The payment facilitator module 124 may determine whether the other parties have approved, at step 924. This may be done successively. In various embodiments, if other parties have approved, then payment facilitator module 124 may withdraw funds from the parties and the patient involved and transfer the funds to a payment requester, at step 926. In various embodiments, the other parties may view user's medical records in the blockchain database 136 to ensure that the patient has received the procedures/treatments. In an example, a use of public-private encryption keys allows an insurer to view general procedures that may take place.

It should be noted that the funds may be withdrawn via the user's mobile wallet. In accordance with various embodiments, if the other parties do not approve, then the payment facilitator may deny the request, at step 918. If the other parties are not required to complete the payment, then the payment facilitator module 124 may follow step 926. This may be done successively. The payment facilitator module 124 may update the payment history blockchain database, at step 928. In various embodiments, hospital C may perform a procedure for patient A. When the procedure is completed, then hospital C may send a request to the patient via the health network application 118 that a predefined amount of money is requested to pay for the procedure. The request is added to the payment blockchain syncing node 128. In various embodiments, if the request already exists, then the request is not added to the payment history blockchain database 134.

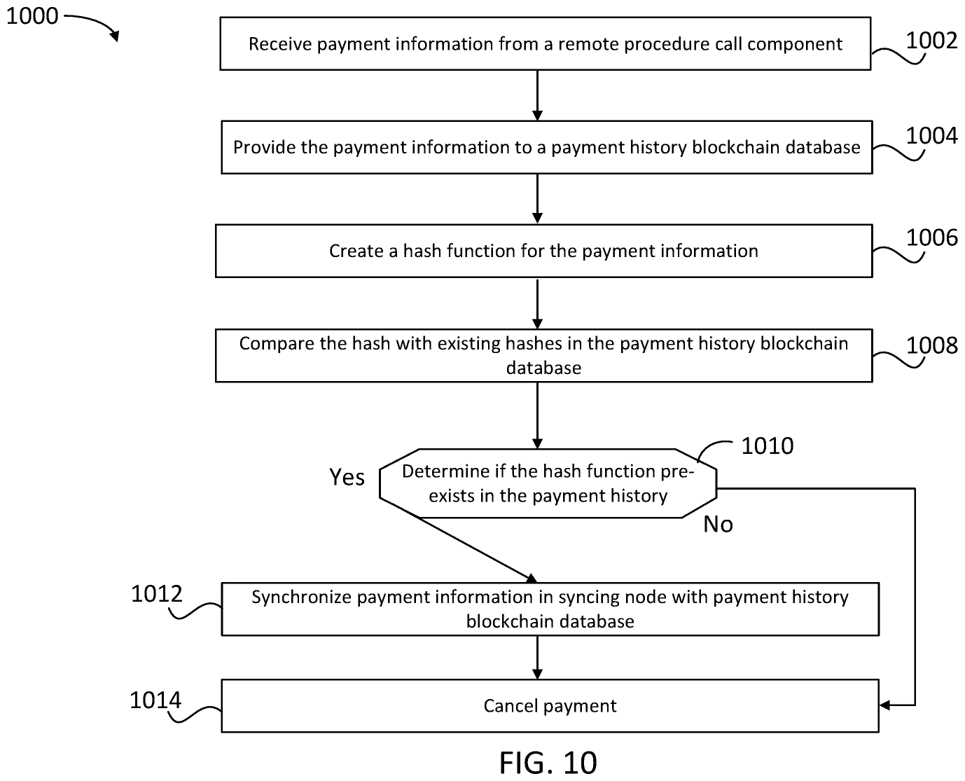

FIG. 10 illustrates a flowchart with steps in a method 1000 for operating the payment blockchain syncing node 128, according to some embodiments. One skilled in the art will appreciate that, for this and other processes and methods disclosed herein, the functions performed in the processes and methods may be implemented in differing order. Furthermore, the outlined steps and operations are only provided as examples, and some of the steps and operations may be optional, combined into fewer steps and operations, or expanded into additional steps and operations without detracting from the essence of the disclosed embodiments.

In various embodiments, the payment blockchain syncing node may receive payment information, e.g., request for a payment, funds have been transferred, and the like, from the RPC component 122, at step 1002. The payment blockchain syncing node 128 may send the payment information to a payment history blockchain database 134, at step 1004. This may be done successively. The payment blockchain syncing node 128 may create a hash function with the payment information for adding the payment information to a payment blockchain syncing node database 128, at step 1006. This may be done successively. The payment blockchain syncing node 128 may compare the hash with existing hashes in the payment history blockchain database, at step 1008. This may be done successively.

In accordance with various embodiments, the payment blockchain syncing node 128 may check the payment history blockchain database 134 to determine if the hash function already exists, e.g., the hash function was previously added to the database and therefore does not need to be added a second time, at step 1010. In various embodiments, if the hash function does not exist, the payment blockchain syncing node 128 may add new information to the payment history blockchain database 134. Further, the payment blockchain syncing node 128 may synchronize information of a payment syncing node with the payment history blockchain database 134, at step 1012. In various embodiments, if the hash function does exist, process is cancelled as the information is already in the payment history blockchain database 134, at step 1014.

### Computer System

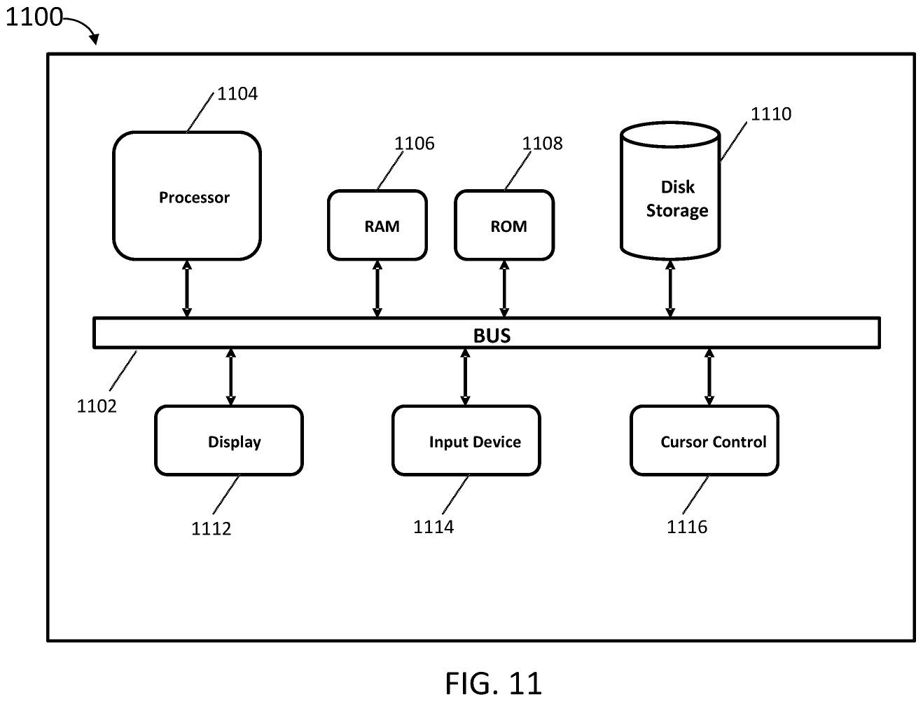

FIG. 11 is a block diagram that illustrates a computer system 1100, upon which embodiments, or portions of the embodiments, of the present teachings may be implemented. In various embodiments of the present teachings, computer system 1100 can include a bus 1102 or other communication mechanism for communicating information, and a processor 1104 coupled with bus 1102 for processing information. In various embodiments, computer system 1100 can also include a memory 1106, which can be a random access memory (RAM) or other dynamic storage device, coupled to bus 1102 for determining instructions to be executed by processor 1104. Memory 1106 also can be used for storing temporary variables or other intermediate information during execution of instructions to be executed by processor 1104. In various embodiments, computer system 1100 can further include a read-only memory (ROM) 1108 or other static storage device coupled to bus 1102 for storing static information and instructions for processor 1104. A storage device 1110, such as a magnetic disk or optical disk, can be provided and coupled to bus 1102 for storing information and instructions.

In various embodiments, computer system 1100 can be coupled via bus 1102 to a display 1112, such as a cathode ray tube (CRT) or liquid crystal display (LCD), for displaying information to a computer user. An input device 1114, including alphanumeric and other keys, can be coupled to bus 1102 for communicating information and command selections to processor 1104. Another type of user input device is a cursor control 1116, such as a mouse, a trackball, or cursor direction keys for communicating direction information and command selections to processor 1104 and for controlling cursor movement on display 1112. This input device 1114 typically has two degrees of freedom in two axes, a first axis (e.g., x) and a second axis (e.g., y), that allows the device to specify positions in a plane. However, it should be understood that input devices 1114 allowing for 3-dimensional (x, y, and z) cursor movement are also contemplated herein.

Consistent with certain implementations of the present teachings, results can be provided by computer system 1100 in response to processor 1104 executing one or more sequences of one or more instructions contained in memory 1106. Such instructions can be read into memory 1106 from another computer-readable medium or computer-readable storage medium, such as storage device 1110. Execution of the sequences of instructions contained in memory 1106 can cause processor 1104 to perform the processes described herein. Alternatively, hard-wired circuitry can be used in place of or in combination with software instructions to implement the present teachings. Thus, implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1104 for execution. Such a medium can take many forms, including but not limited to, non-volatile media, volatile media, and transmission media. Examples of non-volatile media can include, but are not limited to, optical, solid state, and magnetic disks, such as storage device 1110. Examples of volatile media can include, but are not limited to, dynamic memory, such as memory 1106. Examples of transmission media can include, but are not limited to, coaxial cables, copper wire, and fiber optics, including the wires that include bus 1102.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to a computer-readable medium, instructions or data can be provided as signals on transmission media included in a communications apparatus or system to provide sequences of one or more instructions to processor 1104 of computer system 1100 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, and the like.

It should be appreciated that the methodologies described herein including flow charts, diagrams, and accompanying disclosure can be implemented using computer system 1100 as a standalone device or on a distributed network of shared computer processing resources such as a cloud computing network.

In accordance with various embodiments, the systems and methods described herein can be implemented using computer system 1100 as a standalone device or on a distributed network of shared computer processing resources such as a cloud computing network. As such, a non-transitory computer-readable medium can be provided in which a program is stored for causing a computer to perform the disclosed methods for identifying mutually incompatible gene pairs.

It should also be understood that the preceding embodiments can be provided, in whole or in part, as a system of components integrated to perform the methods described. For example, in accordance with various embodiments, the methods described herein can be provided as a system of components or stations for analytically determining novelty responses.

In describing the various embodiments, the specification may have presented a method and/or process as a particular sequence of steps. However, to the extent that the method or process does not rely on the particular order of steps set forth herein, the method or process should not be limited to the particular sequence of steps described. As one of ordinary skill in the art would appreciate, other sequences of steps may be possible. Therefore, the particular order of the steps set forth in the specification should not be construed as limitations on the claims. In addition, the claims directed to the method and/or process should not be limited to the performance of their steps in the order written, and one skilled in the art can readily appreciate that the sequences may be varied and still remain within the spirit and scope of the various embodiments. Similarly, any of the various system embodiments may have been presented as a group of particular components. However, these systems should not be limited to the particular set of components, their specific configuration, communication, and physical orientation with respect to each other. One skilled in the art should readily appreciate that these components can have various configurations and physical orientations (e.g., wholly separate components, units, and subunits of groups of components, different communication regimes between components).

Embodiments disclosed herein include:

A. A computer-implemented method for facilitating payment requests within a health care network includes configuring a patient interface coupled with the health care network for communicating with multiple patients. The computer-implemented method also includes configuring a non-patient interface coupled with the health care network for communicating with multiple non-patients, and receiving a payment request from a payment sender for making a payment to a payment receiver, wherein the payment sender is one and the payment receiver is another of patients and non-patients. The computer-implemented method also includes processing the payment request based on at least one of an authentication of the payment receiver and payment details stored in a blockchain database.

B. A system for facilitating payment requests within a health care network includes a memory circuit storing instructions and one or more processors configured to execute the instructions. The instructions cause the system to configure a patient interface coupled with the health care network for communicating with multiple patients and to configure a non-patient interface coupled with the health care network for communicating with multiple non-patients. The instructions also cause the system to receive a payment request from a payment sender for making a payment to a payment receiver, wherein the payment sender is one and the payment receiver is another of patients and non-patients, and to process the payment request based on at least one of an authentication of the payment receiver and payment details stored in a blockchain database.

C. A computer-implemented method for processing payment information in a healthcare network, includes receiving a payment information from a remote procedure call component. The computer-implemented method also includes providing the payment information to a payment history blockchain database, creating a hash function for the payment information, and comparing the hash function with existing hash functions in the payment history blockchain database. When the hash function matches with an existing hash function in the payment history blockchain database, the computer-implemented method includes synchronizing, in a syncing node, the payment information with payment history blockchain database. The computer-implemented method also includes transferring funds from a payment sender to a payment receiver based on the payment information.

Each one of embodiments A, B, and C may be combined with one or more of the following elements: Element 1, wherein the non-patients include individuals belonging to hospitals, insurance companies, Contract Research Organizations (CROs), and pharmaceutical companies as a payment receiver, and receiving a payment request includes verifying a private key of the individual belonging to the hospital, the insurance companies, the contract Research Organizations, and the pharmaceutical companies based on the blockchain database. Element 2, further including allowing the patients and the non-patients to access the payment details stored in the blockchain database. Element 3, further including identifying at least one of the payment sender or the payment receiver in the blockchain database, and when at least one of the payment sender or the payment receiver is not in the blockchain database, accessing a smart contract to add the payment sender or the payment receiver to the blockchain database. Element 4, wherein processing the payment request includes verifying that a smart contract is present in the blockchain database, the smart contract associating the payment sender and the payment receiver. Element 5, further including updating the blockchain database to reflect that the payment request has been processed. Element 6, further including creating a smart contract for a non-patient when the non-patient requests access to an electronic health record for a patient data, and deleting the smart contract for the non-patient when the patient revokes a permission for the non-patient to access the electronic health record for the patient data. Element 7, further including adding a block for a payment transaction in the blockchain database when the payment request has been successfully processed. Element 8, further including adding a blockchain address for a smart contract to a list of non-patients, wherein the non-patients belong to a hospital or insurance company, contract research organization, or pharmaceutical company when a patient authorizes the list of non-patients to access an electronic health record. Element 9, further including decrypting and loading an electronic health record for one patient to a smart contract previously created for a non-patient when the patient authorizes the non-patient to access the electronic health record.

Each one of embodiments A, B, and C may also be combined with one or more of the following elements: Element 10, wherein the non-patients include individuals belonging to hospitals, insurance companies, Contract Research Organizations (CROs), and pharmaceutical companies as a payment receiver, and to receive a payment request, the one or more processors execute instructions to verify a private key of the individual belonging to the hospital, the insurance companies, the contract Research Organizations, and the pharmaceutical companies based on the blockchain database. Element 11, wherein the one or more processors further execute instructions to allow the patients and the non-patients to access the payment details stored in the blockchain database. Element 12, wherein the one or more processors further execute instructions to identify at least one of the payment sender or the payment receiver in the blockchain database, and when at least one of the payment sender or the payment receiver is not in the blockchain database, to access a smart contract to add the payment sender or the payment receiver to the blockchain database. Element 13, wherein to process the payment request the one or more processors execute instructions to verify that a smart contract is present in the blockchain database, the smart contract associating the payment sender and the payment receiver.

Each one of embodiments A, B, and C may also be combined with one or more of the following elements: Element 14, further including canceling a payment when the hash function is not found in the payment history blockchain database. Element 15, further including adding a new information to the payment history blockchain database when the hash function is not found in the payment history blockchain database. Element 16, wherein the payment information is a payment request, further including requesting approval of the payment request based on a medical record in the healthcare network. Element 17, further including identifying, from the payment information, an identity of a payment sender and an identity of a payment receiver, and verifying that the payment sender and the payment receiver are part of the payment history blockchain database.

It will be appreciated that variants of the above disclosed, and other features and functions or alternatives thereof, may be combined into many other different systems or applications. Presently unforeseen or unanticipated alternatives, modifications, variations, or improvements therein may be subsequently made by those skilled in the art that are also intended to be encompassed by the following claims.

What is claimed is:

1. A computer-implemented method for facilitating payment requests within a health care network, the method comprising:
configuring a patient interface coupled with the health care network for communicating with multiple patients;
configuring a non-patient interface coupled with the health care network for communicating with multiple non-patients;
receiving a payment request from a payment sender for making a payment to a payment receiver, wherein the payment sender is one and the payment receiver is another of patients and non-patients; and
processing the payment request based on at least one of an authentication of the payment receiver and payment details stored in a blockchain database.

2. The computer-implemented method of claim 1, wherein the non-patients include individuals belonging to hospitals, insurance companies, Contract Research Organizations (CROs), and pharmaceutical companies as a payment receiver, and receiving a payment request comprises verifying a private key of the individual belonging to the hospital, the insurance companies, the contract Research Organizations, and the pharmaceutical companies based on the blockchain database.

3. The computer-implemented method of any one of claims 1 and 2, further comprising allowing the patients and the non-patients to access the payment details stored in the blockchain database.

4. The computer-implemented method of any one of claims 1 through 3, further comprising identifying at least one of the payment sender or the payment receiver in the blockchain database, and when at least one of the payment sender or the payment receiver is not in the blockchain database, accessing a smart contract to add the payment sender or the payment receiver to the blockchain database.

5. The computer-implemented method of any one of claims 1 through 4, wherein processing the payment request comprises verifying that a smart contract is present in the blockchain database, the smart contract associating the payment sender and the payment receiver.

6. The computer-implemented method of any one of claims 1 through 5, further comprising updating the blockchain database to reflect that the payment request has been processed.

7. The computer-implemented method of any one of claims 1 through 6, further comprising creating a smart contract for a non-patient when the non-patient requests access to an electronic health record for a patient data, and deleting the smart contract for the non-patient when the patient revokes a permission for the non-patient to access the electronic health record for the patient data.

8. The computer-implemented method of any one of claims 1 through 7, further comprising adding a block for a payment transaction in the blockchain database when the payment request has been successfully processed.

9. The computer-implemented method of any one of claims 1 through 8, further comprising adding a blockchain address for a smart contract to a list of non-patients, wherein the non-patients belong to a hospital or insurance company, contract research organization, or pharmaceutical company when a patient authorizes the list of non-patients to access an electronic health record.

10. The computer-implemented method of any one of claims 1 through 9, further comprising decrypting and loading an electronic health record for one patient to a smart contract previously created for a non-patient when the patient authorizes the non-patient to access the electronic health record.

11. A system for facilitating payment requests within a health care network, the system comprising:
a memory circuit storing instructions; and
one or more processors configured to execute the instructions to cause the system to:
configure a patient interface coupled with the health care network for communicating with multiple patients;
configure a non-patient interface coupled with the health care network for communicating with multiple non-patients;
receive a payment request from a payment sender for making a payment to a payment receiver, wherein the payment sender is one and the payment receiver is another of patients and non-patients; and
process the payment request based on at least one of an authentication of the payment receiver and payment details stored in a blockchain database.

12. The system of claim 11, wherein the non-patients include individuals belonging to hospitals, insurance companies, Contract Research Organizations (CROs), and pharmaceutical companies as a payment receiver, and to receive a payment request the one or more processors execute instructions to verify a private key of the individual belonging to the hospital, the insurance companies, the contract Research Organizations, and the pharmaceutical companies based on the blockchain database.

13. The system of any one of claims 11 and 12, wherein the one or more processors further execute instructions to allow the patients and the non-patients to access the payment details stored in the blockchain database.

14. The system of any one of claims 11 through 13, wherein the one or more processors further execute instructions to identify at least one of the payment sender or the payment receiver in the blockchain database, and when at least one of the payment sender or the payment receiver is not in the blockchain database, to access a smart contract to add the payment sender or the payment receiver to the blockchain database.

15. The system of any one of claims 11 through 14, wherein to process the payment request the one or more processors execute instructions to verify that a smart contract is present in the blockchain database, the smart contract associating the payment sender and the payment receiver.

16. A computer-implemented method for processing payment information in a healthcare network, comprising:
receiving a payment information from a remote procedure call component;
providing the payment information to a payment history blockchain database;
creating a hash function for the payment information;
comparing the hash function with existing hash functions in the payment history blockchain database;
when the hash function matches with an existing hash function in the payment history blockchain database, synchronizing, in a syncing node, the payment information with payment history blockchain database; and
transferring funds from a payment sender to a payment receiver based on the payment information.

17. The computer-implemented method of claim 16, further comprising canceling a payment when the hash function is not found in the payment history blockchain database.

18. The computer-implemented method of any one of claims 16 and 17, further comprising adding a new information to the payment history blockchain database when the hash function is not found in the payment history blockchain database.

19. The computer-implemented method of any one of claims 16 through 18, wherein the payment information is a payment request, further comprising requesting approval of the payment request based on a medical record in the healthcare network.

20. The computer-implemented method of any one of claims 16 through 19, further comprising identifying, from the payment information, an identity of a payment sender and an identity of a payment receiver, and verifying that the payment sender and the payment receiver are part of the payment history blockchain database.
